(19) United States
(12) Patent Application Publication
(10) Pub. No.: US 2014/0018643 A1
(43) Pub. Date: Jan. 16, 2014

(54) SELF-CONTAINED SYSTEM SUITABLE FOR BEING INSERTED INTO AN ANATOMICAL CAVITY

(71) Applicants: Charbel Achkar, Fontenay Sous Bois (FR); Julien Molina, Paris (FR); Yan Haentjens, Bois D'Arcy (FR); Gerard Sou, Ivry Sur Seine (FR); Alain Le Borgne, Velizy Villacoublay (FR); Fabien Koskas, Paris (FR); Georges Alquie, L'Hay Les Roses (FR); Michel Schaller, Le Chesnay (FR)

(72) Inventors: Charbel Achkar, Fontenay Sous Bois (FR); Julien Molina, Paris (FR); Yan Haentjens, Bois D'Arcy (FR); Gerard Sou, Ivry Sur Seine (FR); Alain Le Borgne, Velizy Villacoublay (FR); Fabien Koskas, Paris (FR); Georges Alquie, L'Hay Les Roses (FR); Michel Schaller, Le Chesnay (FR)

(21) Appl. No.: 13/980,569

(22) PCT Filed: Jan. 20, 2012
(86) PCT No.: PCT/EP2012/050846

§ 371 Date: Oct. 1, 2013

(30) Foreign Application Priority Data
Jan. 20, 2011 (FR) 1150462

(51) Int. Cl. A61B 5/07

(52) U.S. Cl. CPC A61B 5/07
USPC 600/302

(57) ABSTRACT

The invention relates to a system (1) suitable for being inserted into an anatomical cavity (30), including: a support structure (10), an antenna (10), an assembly (22) of measuring devices, an assembly (26) for transmitting the data of the measurements carried out by the measuring devices (22), which is suitable for communicating with an external entity (E) by means of the antenna (10), and a device (24) for monitoring the assembly of measuring elements and the transmission assembly (26), said antenna consisting of the support structure (10), and the self-contained system being characterized in that the support structure (10) includes a non-conductive junction (20) supporting the assembly (22) of measuring devices, the transmission assembly (26), and/or the monitoring device (24), wherein the junction is suitable for producing an electrical discontinuity within the support structure (10) forming the antenna while mechanically supporting said support structure (10).

The invention relates generally to the use of sensors to monitor some physiological parameters inside a body, such as blood pressure, body temperature, blood flow, etc.

More precisely, the invention relates to intravascular systems suitable for being inserted into a natural or artificial anatomical cavity and for monitoring such physiological parameters.

Such systems are already known.

For example, document EP 1039 831 discloses an endoluminal graft capable of detecting some parameters such as a volume flow, a speed, etc. The graft comprises a stent on which sensors are installed adapted to communicate with an external entity through an antenna by radiofrequency. The antenna may be composed of the structure of the stent itself, or may be wound around it, and the structure may comprise ceramic junctions in order to break current loops that could be created during radiofrequency coupling.

Furthermore, document U.S. Pat. No. 7,685,762 discloses an endoluminal graft adapted to monitor particularly the blood pressure of a patient. The graft comprises anchoring means, a self-supporting structure adapted to support a capsule carrying sensors, and communicates with an external entity by radiofrequency.

Nevertheless, grafts according to prior art are structurally complex.

Therefore, the invention aims to propose a new graft that has a simpler low cost structure capable of sending information related to internal physiological parameters about a body to an external entity and that can be inserted inside the body by standard placement instruments.

To achieve this, the invention discloses a self-supporting system adapted to be inserted into an anatomical cavity, comprising:
a support structure;
an antenna;
a set of measuring devices;
a transmission assembly for sending data measured using the measuring devices, suitable for communicating with an external entity through the antenna; and
a device for controlling the measuring and transmission elements;

said antenna being composed of the support structure, and the self-supporting system being characterised in that the support structure comprises a non-conducting junction supporting the measuring devices, the transmission assembly and/or the control device and adapted to make electrical discontinuity in the support structure forming the antenna while mechanically maintaining said support structure.

Some preferred but non-limitative aspects of the self-supporting system according to the invention are:
the non-conducting junction is a capsule, and the set of measuring devices, the transmission assembly and/or the control device are housed in said capsule;
the non-conducting junction is made from a biocompatible material;
the non-conducting junction is made from a ceramic material;
the measuring devices comprise at least one of the sensors in the following group: a piezoresistive, piezoelectric, resistive, capacitive, inductive, optical, chemical, biological sensor;
the support structure is a stent;
the support structure is self-expandable;
the transmission assembly communicates with the external entity by radiofrequency, and
the set of measuring devices, the transmission assembly and the control device are integrated into an electronic chip.

According to a second aspect, the invention discloses a device for monitoring at least one item of information related to an anatomical cavity, comprising:
a self-supporting system conforming with the invention; and
an external entity, suitable for remotely querying the self-supporting system.

Some preferred but non-limitative aspects of the device according to the invention are:
the self-supporting system and the external entity are capable of communicating by radiofrequency waves at a frequency of 13.56 MHz, and
the external entity comprises an antenna built into a belt.

Other characteristics, purposes and advantages will become clear after reading the following detailed description with reference to the appended drawings given as non-limitative examples, and on which:

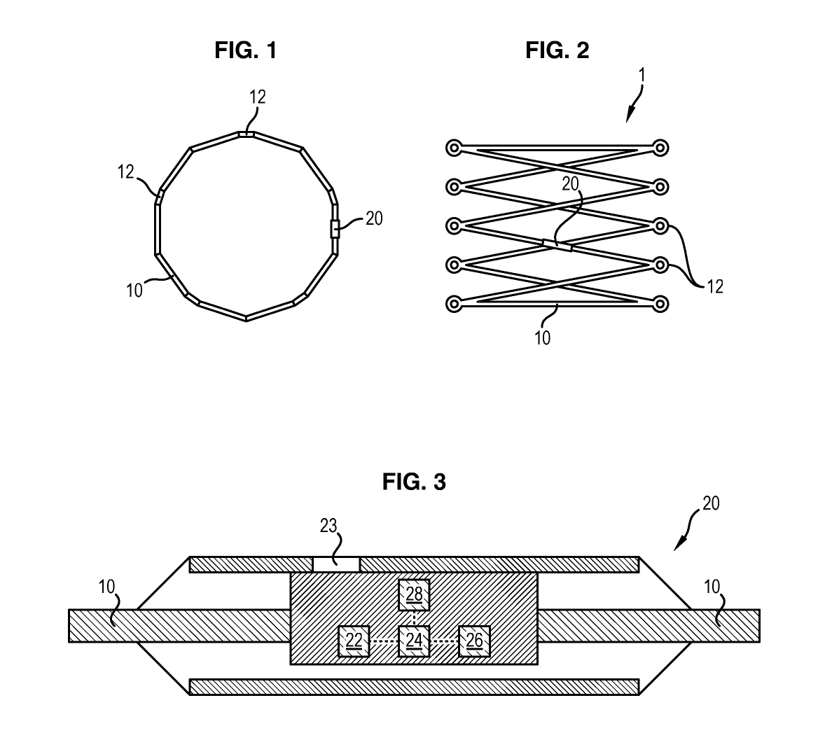

FIG. 1 is a top view of one embodiment of a system according to the invention;

FIG. 2 is a side view of the embodiment in FIG. 1;

FIG. 3 is a sectional view of one embodiment of a non-conducting junction, and

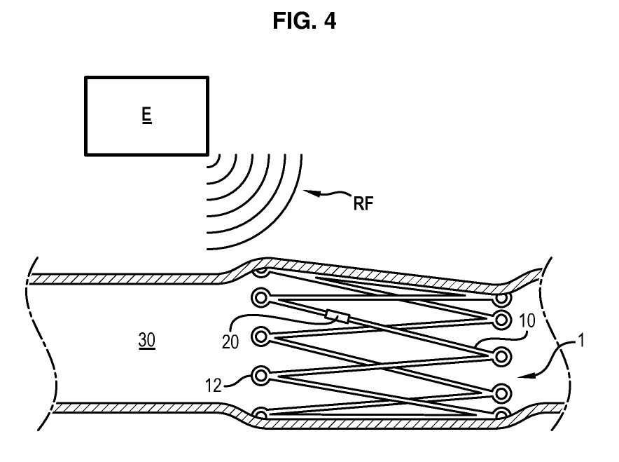

FIG. 4 is a sectional view of a cavity in which the system in FIG. 1 has been expanded.

FIGS. 1 and 2 show a system 1 that comprises a support structure 10, an assembly of measuring devices 22, a transmission assembly 26 for transmitting measurements made by the measurement devices 22, and a device 24 for controlling measurement and transmission assemblies 26.

The support structure 10 is suitable for being inserted, expanded and anchored inside the human body, particularly in a natural or artificial anatomical cavity 30. Typically, natural or artificial anatomical cavities may be part of the cardio-vascular system (artery, vein, heart), the digestive system (oesophagus, stomach, intestine), the ENT system, the urinary system (bladder), genitals (prostate), or the pulmonary system (trachea, bronchea, pleura), while artificial anatomical cavities may be elements of a prosthesis or of any system requiring remote measurements (for example such as extracorporeal blood circulation unit or in a sterile environment).

For example, it may be inserted by means of an appropriate instrument and it may have anchor means that hold it in position in the cavity 30 by applying a radial force on the walls of the cavity 30. For example, it could be a stent, particularly a self-expandable Z stent similar to that described in document EP 0 423 916, that could be installed in the cavity 30 using a conventional placement instrument.

The assembly of measuring devices 22 comprises particularly sensors adapted to measure parameters of interest in the cavity 30. In particular, it may consist of physical measurements (blood flow, blood pressure, temperature, etc.) or chemical parameters (measurement of a glucose content, or the pH, etc.). For example, the assembly 22 may comprise one or several piezoresistive, piezoelectric, resistive, capacitive, inductive, optical, chemical, biological sensors, etc., for example like a pressure sensor, a temperature sensor, an ultrasound sensor, a pH sensor, an accelerometer, an infrared detector, an electrical potential sensor, an optical sensor, an immunological sensor, an oxygen detector, a comparative genomic hybridization chip (ARRAY chip) or a DNA (DeoxyriboNucleic Acid) chip.

These parameters are then transmitted to the control device 24 that is suitable for managing firstly the assembly of measuring devices 22 and secondly the transmission assembly 26.

The control device 24 may be an electronic system comprising an analogue-digital converter suitable for converting physiological measurements at the output from the sensors (analogue data) into digital data (coded as a function of the electrical level of the analogue data). These digital data are conditioned by a digital processing unit and are then transmitted on a wireless link to an external entity E, typically an external monitoring device, through the measurement transmission assembly and an antenna.

The measurement transmission assembly comprises at least a transmitter suitable for transmitting information on a wireless system to the external entity E through the antenna, and a receiver suitable for receiving information by radiofrequency RF waves from the external entity E, through the antenna.

The wireless system can for example operate by radiofrequency RF waves. In particular, it may use RFID (Radio Frequency IDentification), or Bluetooth, Wi-Fi, Zegbee communications, etc.

The electronic system 24, the transmitter, the receiver 26 and possibly all or some of the sensors 22 may be built into one or several electronic chips. The chip may also conventionally comprise a microcontroller adapted to manage the communication protocol and collisions, to encrypt/decrypt data, etc., a storage system of the EEPROM (Electrically-Erasable Programmable Read-Only Memory) type so as to temporarily save data transmitted by the various sensors before sending them to the external entity E, etc.

The external entity E comprises a read terminal among other devices, suitable for communicating with the electronic system 24 onboard the system 1.

Transmission and reception of data by radiofrequency RF waves will not be described in more detail in the following because they are known to those skilled in the art. In particular, they satisfy the various existing standards, such as standard ISO/IEC 14443 or standard ISO/IEC 15693. Standard ISO/IEC 14443 will be preferred for this invention because the modulation described in this standard is more suitable for a remote-power supply, it provides an identifier specific to the medical field and it performs collision processing, such that it is possible to install several electronic chips at the same time, and therefore manage several systems 1 conforming with the invention at the same time in the body.

The antenna in this invention is composed of all or part of the support structure 10 that is then made from a conducting material such as steel. It is connected to the transmitter and the receiver 26, to enable communication between the electronic system 24 and the external entity E.

The antenna 10 may also be used to recover energy from the radiofrequency RF waves and supply all or some of the sensors and the electronic system, thus reducing the size of the system 1. To achieve this, the system 1 also comprises an energy accumulator 28 housed for example within the electronic chip, to recover electrical energy from the radiofrequency RF waves. The energy thus recovered is then stored and distributed to the various components in the system 1 that then becomes a battery-free communication terminal.

The external entity E also comprises a communication terminal to dialog with the onboard electronic system 24 by radio waves, to transmit the energy necessary to supply the energy accumulator 28 with the energy necessary to supply the measurement assembly 22, the transmission assembly 26 and the electronic system 24.

The support structure 10 also comprises a non-conducting junction 20, such that the structure 10 forms a discontinuous metallic loop in order to prevent the formation of current loops that could reduce the efficiency of radiofrequency coupling with the external entity E.

The non-conducting junction 20 is made from an electrically insulating and biocompatible material (which may be for example conforming with standards in force, particularly standard NF EN 10993 on biological assessment of medical devices), with a good resistance to mechanical constraints. Depending on its location, the material may also be resistant to aggressions from its environment, for example it may prevent cellular bonding, it may be antithrombotic, and/or be resistant to steam (in other words resistant to steam at 121° C. for about twenty minutes).

For example, the junction 20 may be made from ceramic (particularly zirconium).

Furthermore, according to the embodiment shown in FIGS. 1 and 2, the non-conducting junction 20 is in the form of a capsule and it houses some or all of the sensors 22, the transmission assembly 26, the electronic system 24 and possibly the energy accumulator 28. The non-conducting junction 20 therefore plays a multiple role in this case, and particularly:
mechanical retaining of the support structure 10 while the system 1 is being anchored in the cavity 30,
mechanical continuity and electrical discontinuity in the metallic structure of the support structure 10, so as to transform it into a radiating element and to be able to use it as an antenna;
support and protection of the sensor assembly 22, the electronic system 24 and the transmission assembly 26;
enable orientation of the sensor inside the cavity 30 as a function of the type of sensor used for the studied phenomenon. Typically, for a pressure sensor, the junction 20 is oriented such that a pressure measurement window 23 is directed towards the inside of the cavity 30;
maintain the connection between the sensor assembly 22, the electronic system and the transmission assembly 26 to the antenna 10.

Therefore, it is used to simply and economically connect the elements forming the system 1 and make them functional, to obtain a platform for integration of electronic microsystems with wireless communication (in this case by radiofrequency).

Furthermore, the non-conducting junction 20 may be made by coating the different elements housed in it in order to guarantee their protection, or it may be hollow and comprise a removable cover for access to its contents.

Finally, the non-conducting junction 20 may have tapered ends, as shown in the appended FIG. 3, possibly made from the same material as the support structure 10, and holding the elements that are housed in it and electrically connecting them to the structure 10.

When the support structure 10 is used as an antenna, its dimensions may influence the choice of the transmission frequency, the signal quality and the transmission distance, and the transferable energy quantity.

As a first approximation, the support structure 10 may be considered as being an inductive loop with a diameter varying from a few millimeters to a few centimetres. The natural frequency of the loop measured by opening the support structure 10 at a point in order to open the loop, is the maximum absolute frequency at which the loop can be used as an inductive antenna. Therefore if the support structure 10 is to be used as an inductive antenna, the transmission frequency has to be a few hundred MHz, considering the dimensions of this structure.

However, the device can operate at higher frequencies provided that the support structure 10 is broken down into several electrically discontinuous strands connected to each other. As a variant, each structure 10 may comprise eyelets 12 adapted to cooperate with eyelets in a similar system in order to form a longer system.

The environment in which the radiofrequency RF waves have to propagate may also have an influence on the choice of the transmission frequency. Considering that system 1 has to be placed deep inside the human body, the signal must pass through up to about ten centimetres of human tissue, composed largely of water (70%). It is preferable to use a frequency of less than 30 MHz, so that the transmission frequency is not attenuated by the surroundings; this corresponds to the frequency range for which attenuation of the signal due to water remains acceptable.

Considering the above, frequency ranges adapted for communication by radiofrequency may for example be the 134 kHz and 13.56 MHz ranges, conforming with existing ISM (International Safety Management) standards. Considering the security and confidentiality necessary for this type of application, it would be possible for example to use the 13.56 MHz frequency that enables data flows (and particularly encrypted data flows) much higher than data flows obtained at 134 kHz.

It can be seen that despite the use of the support structure 10 as antenna and the presence of the non-conducting junction 20 on structure 10, the structure maintains its mechanical properties. For example, when the support structure 10 is a self-expandable stent, it maintains its capability of being automatically fixed to the walls of the cavity and remains retractable. Therefore, it can be placed and removed using the same instruments as for a comparable self-expandable stent without a non-conducting junction. The system 1 may be located in the cavity 30 temporarily or permanently. In the case of temporary use, a recovery wire is passed through the eyelets 12 of the support structure 10. The use of an appropriate removal instrument makes it possible to compress the support structure 10 so as to insert it in a tube with a corresponding diameter and withdraw the system 1 from the cavity 30. Refer to the description in document EP 0 423 916 for further information about placement and removal of the system 1.

The antenna of the external entity E may be in the form of a metallic loop that can be carried at the waist or the chest of a patient. For example, it may be used for remote power supply to the system 1 using the support structure 10 as inductive antenna by electromagnetic coupling at 13.56 MHz.

As a variant, the external entity E may be interfaced with a medical system (for example connected to the personal medical file).

Obviously, this invention is in no way limited to the embodiment described above and shown on the drawings, but those skilled in the art would be capable of making many variants and modifications to it.

1. Self-supporting system adapted to be inserted into an anatomical cavity, comprising:
a support structure;
an antenna;
a set of measuring devices;
a transmission assembly for sending data measured using the measuring devices, suitable for communicating with an external entity through the antenna; and
a device for controlling the measuring and transmission elements;
said antenna being composed of the support structure, and
wherein the support structure comprises a non-conducting junction supporting the measuring devices, the transmission assembly and/or the control device, and adapted to make an electrical discontinuity in the support structure forming the antenna while mechanically maintaining said support structure.

2. Self-supporting system according to claim 1, in which the non-conducting junction is a capsule, and the set of measuring devices, the transmission assembly and/or the control device are housed in said capsule.

3. Self-supporting system according to claim 1, in which the non-conducting junction is made from a biocompatible material.

4. Self-supporting system according to claim 1, in which the non-conducting junction is made from a ceramic material.

5. Self-supporting system according to claim 1, in which the measuring devices comprise at least one of the sensors in the following group: a piezoresistive, piezoelectric, resistive, capacitive, inductive, optical, chemical, biological sensor.

6. Self-supporting system according to claim 1, in which the support structure is a stent.

7. Self-supporting system according claim 1, in which the support structure is self-expandable.

8. Self-supporting system according to claim 1, in which the transmission assembly communicates with the external entity by radiofrequency.

9. Self-supporting system according to claim 1, in which the set of measuring devices, the transmission assembly and the control device are integrated into at least one electronic chip.

10. Device for monitoring at least one item of information related to an anatomical cavity, characterised in that it comprises:
a self-supporting system according to claim 1; and
an external entity, suitable for remotely querying the self-supporting system.

11. Device according to claim 10, in which the self-supporting system and the external entity are capable of communicating by radiofrequency waves at a frequency of 13.56 MHz.

12. Device according to claim 10, in which the external entity comprises an antenna built into a belt.
